(19) United States
(12) Patent Application Publication
(10) Pub. No.: US 2025/0176873 A1
(43) Pub. Date: Jun. 5, 2025

(54) NON-CONTACT OXYGEN SATURATION ESTIMATION USING AMBIENT LIGHT

(71) Applicant: DETECTIVIO AB, Göteborg (SE)

(72) Inventor: Taha KHAN, Halmstad (SE)

(21) Appl. No.: 18/841,078

(22) PCT Filed: Feb. 24, 2023
(86) PCT No.: PCT/SE2023/050166

§ 371 Date: Aug. 23, 2024

(30) Foreign Application Priority Data
Feb. 25, 2022 (SE) 2250253-8

(51) Int. Cl. A61B 5/1455; A61B 5/00

(52) U.S. Cl. CPC A61B 5/14551; A61B 5/7225; A61B 5/725; A61B 5/7267; A61B 5/7275

(57) ABSTRACT

A non-contact estimation of oxygen saturation comprises pre-processing a PPG signal of light reflected from a skin of a subject illuminated by ambient light by filtering the PPG signal to obtain a smoothed pulse signal. A plurality of frequency domain and time domain features are extracted from the smoothed pulse signal and statistical parameters are computed of the time domain features. Oxygen saturation is estimated for the subject based on the frequency domain features and the statistical parameters of the time domain features and an oxygen saturation estimation model trained for estimating oxygen saturation based on input frequency domain features and input statistical parameters of time domain features.

### TECHNICAL FIELD

The present invention generally relates to oxygen saturation estimation, and in particular to a method and a system for non-contact estimation of oxygen saturation and a method for generating an oxygen saturation estimation model useful in such non-contact estimation of oxygen saturation.

### BACKGROUND

Oxygen saturation is the fraction of oxygen-saturated hemoglobin relative to total hemoglobin (unsaturated+saturated) in the blood. The human body requires and regulates a very precise and specific balance of oxygen in the blood. Normal arterial blood oxygen saturation (SaO₂) levels in humans are 95-100%. If the level is below 90%, it is considered low and called hypoxemia. Arterial blood oxygen levels below 80% may compromise organ function, such as the brain and heart. Continued low oxygen levels may lead to respiratory or cardiac arrest.

Oxygen saturation can be measured in different tissues, including arterial oxygen saturation (SaO₂) as determined by arterial blood gas test, venous oxygen saturation (SvO₂) typically used under treatment with a heart lung machine (extracorporeal circulation), tissue oxygen saturation (StO₂) measured by near infrared spectroscopy and peripheral oxygen saturation (SpO₂), which is an approximation of SaO₂ usually measured by a pulse oximeter device. SpO₂ can be calculated with pulse oximetry according to the formula:

$${Sp O}_{2} = \frac{{Hb O}_{2}}{{Hb O}_{2} + Hb}$$

where HbO₂ is oxygenated hemoglobin (oxyhemoglobin) and Hb is deoxygenated hemoglobin. The pulse oximeter consists of a small device that clips to the body (typically a finger, an earlobe or an infant's foot) and transfers its readings to a reading meter by wire or wirelessly. The pulse oximeter uses light-emitting diodes of different wavelengths in conjunction with a light-sensitive sensor to measure the absorption of red and infrared light in the extremity. The difference in absorption between oxygenated and deoxygenated hemoglobin makes the calculation possible according to the above presented formula.

There is, though, a need for more convenient measurements of oxygen saturation, and in particular for non-contact oxygen saturation measurements that do not require attaching or connecting any measurement equipment to the body of a subject.

U.S. Pat. No. 11,103,144 discloses a method of measuring a physiological parameter, such as oxygen saturation level, in a contactless manner. The method includes acquiring a plurality of image frames for a subject, acquiring a first color channel value, a second color channel value, and a third color channel value for at least one image frame included in the plurality of image frames. The method further includes calculating a first difference and a second difference on the basis of the first color channel value, the second color channel value, and the third color channel value for at least one image frame included in the plurality of image frames. The first difference represents a difference between the first color channel value and the second color channel value for the same image frame, and the second difference represents a difference between the first color channel value and the third color channel value for the same image frame.

U.S. Pat. No. 10,888,280 discloses a photoplethysmography (PPG) circuit that obtains PPG signals at a plurality of wavelengths. A signal processing module obtains at least a first spectral response around a first wavelength and a second spectral response around a second wavelength. The signal processing device generates PPG input data using the PPG signals. The PPG input data includes one or more parameters obtained from each of the first spectral response and the second spectral response. A neural network processing device generates an input vector including the PPG input data and determines an output vector including health data. The health data includes an oxygen saturation level, a glucose level or a blood alcohol level.

### SUMMARY

It is general objective to provide a non-contact oxygen saturation estimation that does not require special lighting conditions.

This and other objectives are met by embodiments of the invention.

The present invention is defined in the independent claims. Further embodiments of the invention are defined in the dependent claims.

An aspect of the invention relates to a method for non-contact estimation of oxygen saturation. The method comprises pre-processing a photoplethysmography (PPG) signal of light reflected from a skin of a subject illuminated by ambient light by filtering the PPG signal to obtain a smoothed pulse signal. The method also comprises extracting a plurality of frequency domain and time domain features from the smoothed pulse signal by extracting time domain features from the smoothed pulse signal with respect to time and extracting frequency domain features from the smoothed pulse signal with respect to frequency. The method additionally comprises computing statistical parameters of the time domain features. The statistical parameters represent measured quantities of a statistical population describing the respective time domain features. The method further comprises estimating oxygen saturation for the subject based on the frequency domain features and the statistical parameters of the time domain features and an oxygen saturation estimation model trained for estimating oxygen saturation based on input frequency domain features and input statistical parameters of time domain features.

Another aspect of the invention relates to computer-implemented method of generating an oxygen saturation estimation model. The method comprises pre-processing a plurality of PPG signals of light reflected from skins of a plurality of subjects illuminated by ambient light by filtering the PPG signals to obtain a plurality of smoothed pulse signals. The method also comprises extracting, from each smoothed pulse signal of the plurality of smoothed pulse signals, a plurality of frequency domain and time domain features from the smoothed pulse signal by extracting time domain features from the smoothed pulse signal with respect to time and extracting frequency domain features from the smoothed pulse signal with respect to frequency. The method additionally comprises computing statistical parameters of the time domain features. The statistical parameters represent measured quantities of a statistical population describing the respective time domain features. The method further comprises training the oxygen saturation estimation model based on the frequency domain features and the statistical parameters of the time domain features and actual oxygen saturation values obtained for the plurality of subjects.

A further aspect of the invention relates to a non-transitory computer-readable medium storing instructions that, when executed by a processor, cause the processor to pre-process a PPG signal of light reflected from a skin of a subject illuminated by ambient light by filtering the PPG signal to obtain a smoothed pulse signal. The processor is also caused to extract a plurality of frequency domain and time domain features from the smoothed pulse signal by extracting time domain features from the smoothed pulse signal with respect to time and extracting frequency domain features from the smoothed pulse signal with respect to frequency. The processor is additionally caused to compute statistical parameters of the time domain features. The statistical parameters represent measured quantities of a statistical population describing the respective time domain features. The processor is further caused to estimate oxygen saturation for the subject based on the frequency domain features and the statistical parameters of the time domain features and an oxygen saturation estimation model trained for estimating oxygen saturation based on input frequency domain features and input statistical parameters of time domain features.

Yet another aspect of the invention relates to a non-transitory computer-readable medium storing instructions that, when executed by a processor, cause the processor to pre-process a plurality of PPG signals of light reflected from skins of a plurality of subjects illuminated by ambient light by filtering the PPG signals to obtain a plurality of smoothed pulse signals. The processor is also caused to extract, from each smoothed pulse signal of the plurality of smoothed pulse signals, a plurality of frequency domain and time domain features from the smoothed pulse signal by extracting time domain features from the smoothed pulse signal with respect to time and extracting frequency domain features from the smoothed pulse signal with respect to frequency. The processor is additionally caused to compute statistical parameters of the time domain features. The statistical parameters represent measured quantities of a statistical population describing the respective time domain features. The processor is further caused to train an oxygen saturation estimation model based on the frequency domain features and the statistical parameters of the time domain features and actual oxygen saturation values obtained for the plurality of subjects.

An aspect of the invention relates to a system for non-contact estimation of oxygen saturation. The system comprises a camera configured to record a PPG signal of light reflected from a skin of a subject illuminated by ambient light, The system also comprises at least one memory configured to store an oxygen saturation estimation model trained for estimating oxygen saturation based on input frequency domain features and input statistical parameters of the domain features and store the PPG signal recorded by the camera. The system further comprises at least one processor configured to pre-process the PPG signal by filtering the PPG signal to obtain a smoothed pulse signal. The at least one processor is also configured to extract a plurality of frequency domain and time domain features from the smoothed pulse signal by extracting time domain features from the smoothed pulse signal with respect to time and extracting frequency domain features from the smoothed pulse signal with respect to frequency. The processor is additionally caused to compute statistical parameters of the time domain features. The statistical parameters represent measured quantities of a statistical population describing the respective time domain features. The at least one processor is further configured to estimate oxygen saturation for the subject based on the frequency domain features and the statistical parameters of the time domain features and the oxygen saturation estimation model stored in the at least one memory.

The present invention enables non-contact or contactless estimation of oxygen saturation without the need for special lighting, such as a dedicated infrared light source. In clear contrast, contactless estimation of oxygen saturation can be conducted in ambient light conditions. Hence, no dedicated light source with special light spectrum is needed as ambient light sources and even daylight could be used as “light source” when conducting the contactless oxygen saturation estimation.

### BRIEF DESCRIPTION OF THE DRAWINGS

The embodiments, together with further objects and advantages thereof, may best be understood by making reference to the following description taken together with the accompanying drawings, in which:

FIG. 1 are diagrams illustrating pre-processing of a photoplethysmography (PPG) signal. (A) raw PPG signal, (B) PPG signal smoothed using a median filter, (C) smoothed PPG signal filtered using a 3-standard deviation filter, (D) truncated PPG signal, and (E) final PPG pulse signal smoothed using a moving average filter.

FIG. 2 illustrates time domain features for estimating oxygen saturation.

FIG. 3 schematically illustrates a regression-based random forest algorithm.

FIG. 4 is a diagram illustrating feature permutation scores when training the random forest model for predicting oxygen saturation.

FIG. 5 is a diagram illustrating a Leave one out cross-validation of oxygen saturation estimation (full arrow represents actuation SpO₂ signal and hatched arrow represents estimated SpO₂ signal).

FIG. 6 is a flow chart illustrating a method for non-contact estimation of oxygen saturation according to an embodiment.

FIG. 7 is a flow chart illustrating a computer-implemented method of generating an oxygen saturation estimation model according to an embodiment.

FIG. 8 is a flow chart illustrating an additional, optional step of the method shown in FIG. 7.

FIG. 9 is a flow chart illustrating an embodiment of the additional, optional step of FIG. 8.

FIG. 10 is a flow chart illustrating various embodiments of the pre-processing step in the methods shown in FIGS. 6 and 7.

FIG. 11 is a schematic illustration of a device configured to generate an oxygen saturation estimation model according to an embodiment.

FIG. 12 is a schematic illustration of a device configured to non-contact estimate oxygen saturation and/or generation of an oxygen saturation estimation model according to an embodiment.

FIG. 13 is a schematic illustration of a system for non-contact estimation of oxygen saturation according to an embodiment.

### DETAILED DESCRIPTION

The present invention generally relates to oxygen saturation estimation, and in particular to a method and a system for non-contact estimation of oxygen saturation and a method for generating an oxygen saturation estimation model useful in such non-contact estimation of oxygen saturation.

The current techniques for estimating oxygen saturation in a subject, typically a human subject, are either contact-dependent techniques or require special measurement conditions. The contact-dependent techniques use a pulse oximeter device clipped to a body extremity of the subject to perform the oxygen saturation estimations by measuring absorption of red and infrared light in the body extremity. Contactless techniques have been proposed in the art to estimate tissue oxygen saturation (StO₂) by near infrared (NIR) spectroscopy. These contactless techniques therefore require the presence of an infrared light source in order to perform the StO₂ measurements.

The present invention enables contactless estimation of oxygen saturation but does not require the presence of a dedicated infrared light source. In clear contrast, the oxygen saturation estimation of the invention can be conducted in ambient light conditions. Hence, no dedicated light source with special light spectrum is needed as ambient light sources and even daylight could be used as “light source” when conducting the oxygen saturation estimation.

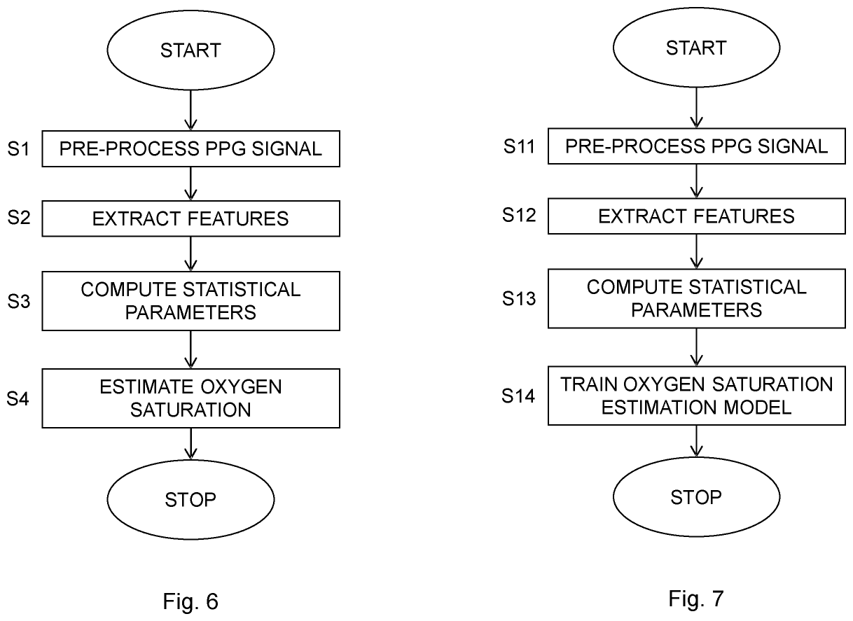

An aspect of the invention relates to a method for non-contact estimation of oxygen saturation, see FIG. 6. The method comprises pre-processing, in step S1, a photoplethysmography (PPG) signal of light reflected from a skin of a subject illuminated by ambient light by filtering the PPG signal to obtain a smoothed pulse signal. A next step S2 comprises extracting a plurality of frequency domain and time domain features from the smoothed pulse signal by extracting time domain features from the smoothed pulse signal with respect to time and extracting frequency domain features from the smoothed pulse signal with respect to frequency. Statistical parameters of the time domain features are computed in step S3. The statistical parameters represent measured quantities of a statistical population describing the respective time domain features. The method also comprises estimating oxygen saturation for the subject in step S4 and based on the frequency domain features and the statistical parameters of the time domain features and an oxygen estimation model. According to the invention, the oxygen estimation model is trained for estimating oxygen saturation based on input frequency domain features and input statistical parameters of time domain features.

PPG is a non-invasive optical method that measures volumetric variations of blood circulation representing blood volume changes in the microvascular bed of the monitored tissue of the subject. According to the invention, the PPG signal is of light reflected from the skin of the subject illuminated by ambient light. The ambient light is preferably ambient visible light, i.e., light having wavelengths in the range of 400 to 700 nm. The ambient light illuminating the skin of the subject could be from one or more light sources or lamps present in the room or facility where the oxygen saturation estimation is conducted. The at least one light source could, for instance, be one or more light sources arranged in the ceiling, one or more light sources arranged at a wall and/or one or more stand-alone light sources. The at least one light source is not arranged to specifically illuminate the subject but merely to provide background or ambient illumination. The present invention is, however, not limited to having one or more light sources for conducting the non-contact estimation of oxygen saturation. In clear contrast, daylight from one or more windows could be sufficient as ambient light illuminating the skin of the subject.

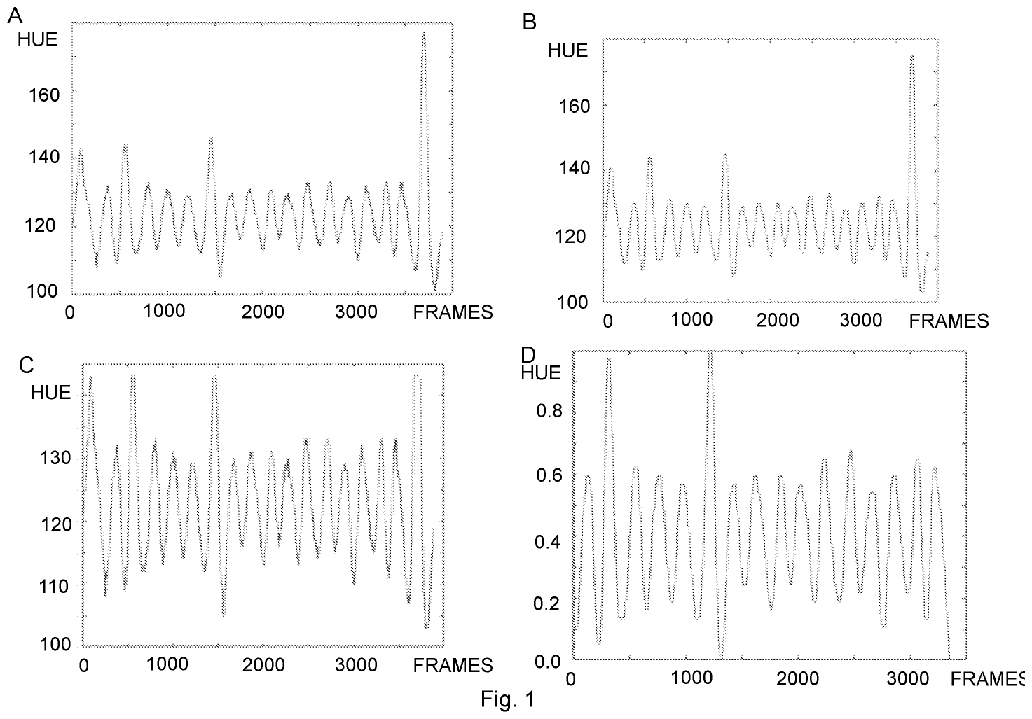

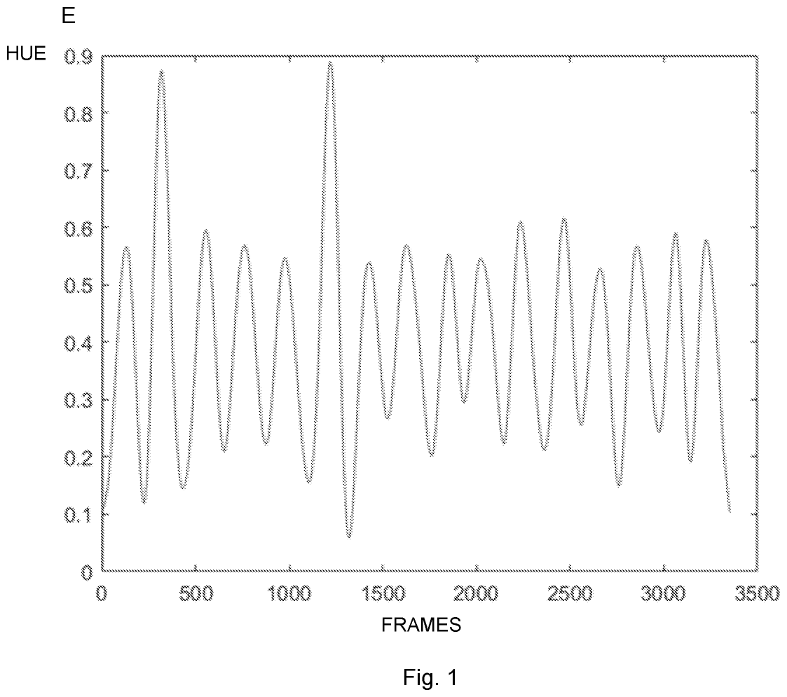

The PPG signal is pre-processed in step S1 by filtering the PPG signal to obtain a smoothed pulse signal. FIG. 1A is an example of a raw PPG signal of light reflected from the skin of a human subject illuminated by ambient light. FIGS. 1B to 1E illustrate various examples of pre-processed PPG signals according to embodiments.

Frequency domain and time domain features are then extracted in step S2 from the smoothed pulse signal obtained in step S1. Time domain features are features extracted from the smoothed pulse signal with respect to time. Frequency domain features are features extracted from the smoothed pulse signal with respect to frequency rather than time. Illustrative, but non-limiting examples, of time domain features are given in Table 1 and frequency domain features are given in Table 2. A time-domain graph of the smoothed pulse signal indicates how the signal changes with time, whereas a frequency-domain graph of the smoothed pulse signal shows how much of the signal lies within each given frequency band over a range of frequencies.

Statistical parameters are then computed in step S3 of the time domain features. These statistical parameters represent measured quantities of a statistical population that summarizes or describes an aspect of the respective time domain features. Statistical population as used herein means multiple, i.e., at least two, statistical parameters that describe a time domain feature. Illustrative, but non-limiting, examples of such statistical parameters include mean (or average), median, standard deviation, mean (or average) absolute deviation and interquartile range (IQR).

The statistical parameters of the time domain features as computed in step S3 and the frequency domain features extracted in step S2 are input into an oxygen saturation estimation model in step S4 to estimate the oxygen saturation for the subject. The oxygen saturation estimation model has been trained for estimating oxygen saturation based on input frequency domain features and input statistical parameters of time domain features, which is further described herein in connection with FIG. 7. Hence, the non-contact estimation of oxygen saturation uses an oxygen saturation estimation model that outputs an estimate of oxygen saturation given input frequency domain features and statistical parameters of time domain features.

FIG. 7 is a flow chart illustrating a computer-implemented (CI) method of generating an oxygen saturation model. The method comprises pre-processing, in step S11, a plurality of PPG signals of light reflected from skins of a plurality of subjects illuminated by ambient light by filtering the PPG signals to obtain a plurality of smoothed pulse signals. A next step S12 comprises extracting, from each smoothed pulse signal of the plurality of smoothed pulse signals, a plurality of frequency domain features and time domain features by extracting time domain features from the smoothed pulse signal with respect to time and extracting frequency domain features from the smoothed pulse signal with respect to frequency. Statistical parameters are computed in step S13 of the time domain features extracted in step S12. The statistical parameters represent measured quantities of a statistical population describing the respective time domain features. The oxygen saturation estimation model is then trained in step S14 based on the frequency domain features and the statistical parameters of the time domain features and actual oxygen saturation values obtained for the plurality of subjects.

Thus, an oxygen saturation estimation model is trained based features extracted from pre-processed PPG signals obtained for different subjects. Respective features domain features and statistical parameters of time domain features are determined for each of the PPG signals and therefore for the different subjects. For instance, step S12 could comprise extracting, for each smoothed pulse signal obtained in step S11, a set of plurality of frequency domain features and time domain features. This means that a plurality of such sets of features are extracted from the smoothed pulse signal in step S12, and more preferably one set of features per smoothed pulse signal and subject. Correspondingly, a plurality of sets of frequency domain features and statistical parameters of time domain features are obtained following the computations in step S13. The oxygen saturation estimation model is then trained using the plurality of sets of frequency domain features and statistical parameters of time domain features and the actual oxygen saturation values of the subjects in step S14. The training in step S14 thereby learns the oxygen saturation estimation model to correlate the frequency domain features and statistical parameters of time domain features with oxygen saturation values.

The oxygen saturation estimation model can be trained in FIG. 7 to accurately estimate oxygen saturation of a subject based on a processing of a PPG signal of light reflected from the skin of the subject illuminated merely by ambient light. Thus, by providing a number of such PPG signals from various subjects, the oxygen saturation estimation model will learn how changes in the PPG signals, as represented by the extracted frequency domain features and the computed statistical parameters of the extracted time domain features, reflect changes in oxygen saturation.

The actual oxygen saturation values input to the oxygen saturation estimation model during the training step S14 are preferably measured according to well-known oxygen saturation methods or techniques, for instance pulse oximetry measurements using a pulse oximeter device.

The oxygen saturation estimation model may be implemented according to various embodiments. For instance, the oxygen saturation estimation model is a computer-implemented oxygen saturation model and could be in the form a machine learning (ML) model. Generally, ML algorithms build a mathematical model based on training data, i.e., input frequency domain features and statistical parameters of time domain features according to the invention, in order to make predictions or decisions without being explicitly programmed to do so. There are various types of ML algorithms that differ in their approach, the type of data they input and output, and the type of task or problem that they are intended to solve. Illustrative, but non-limiting, examples of such ML algorithms include supervised learning algorithms, unsupervised learning algorithms, semi-supervised learning algorithms, reinforcement learning algorithms, self-learning algorithms, feature learning algorithms, sparse dictionary learning algorithms, anomaly detection algorithms, and association rule learning algorithms.

Performing machine learning involves creating a model, which is trained on training data and can then process additional data to make predictions or decisions. Various types of ML models could be used according to the embodiments, including, but not-limited to artificial neural networks, decision trees, support vector machines, regression analysis, Bayesian networks and Genetic algorithms.

Furthermore, deep learning, also known as deep structured learning, is a ML method based on artificial neural networks with representation learning. Learning can be supervised, semi-supervised or unsupervised. Deep learning architectures, such as deep neural networks, deep belief networks, recurrent neural networks and convolutional neural networks, could be used to train and implement the oxygen saturation estimation model. “Deep” in deep learning comes from the use of multiple layers in the network. Deep learning is concerned with an unbounded number of layers of bounded size, which permits practical application and optimized implementation, while retaining theoretical universality under mild conditions. In deep learning the layers are also permitted to be heterogeneous and to deviate widely from biologically informed connectionist models, for the sake of efficiency, trainability and understandability.

Hence, in an embodiment, step S14 in FIG. 7 comprises training an oxygen saturation estimation ML model, such as a random forest (RF) based oxygen saturation model. Hence, in a preferred embodiment, the oxygen saturation estimation model trained in step S14 of FIG. 7 and used in step S4 in FIG. 6 is preferably a RF-based oxygen saturation model.

Random forests or random decision forests are an ensemble learning method for classification, regression and other tasks that operates by constructing a multitude of decision trees at training time. For classification tasks, the output of the random forest is the class selected by most trees. For regression tasks, the mean or average prediction of the individual trees is returned. Random decision forests correct for decision trees' habit of overfitting to their training set. Random forests generally outperform decision trees.

Hence, by using multiple decision trees for prediction, the RF-based oxygen saturation estimation model eliminates prediction bias that occurs if a single decision tree is used for decision making. Also, the random selection of data for training and testing reduces variance in the data that prevents overfitting.

Another advantage of using the RF algorithm is that it performs feature selection during training. Features that are most correlated with the training targets are selected by the RF algorithm using permutation scores. RF permutes feature values to estimate if the permutation deteriorates the prediction performance compared to a baseline. The features that are not correlated show no changes when the values are permutated suggesting that there is no difference between the permuted values and the original sequence of values. This suggests that the feature is a noise that does not contribute to training and can be discarded. On the other hand, the permutation of features that are correlated with the training targets results in reducing the prediction accuracy.

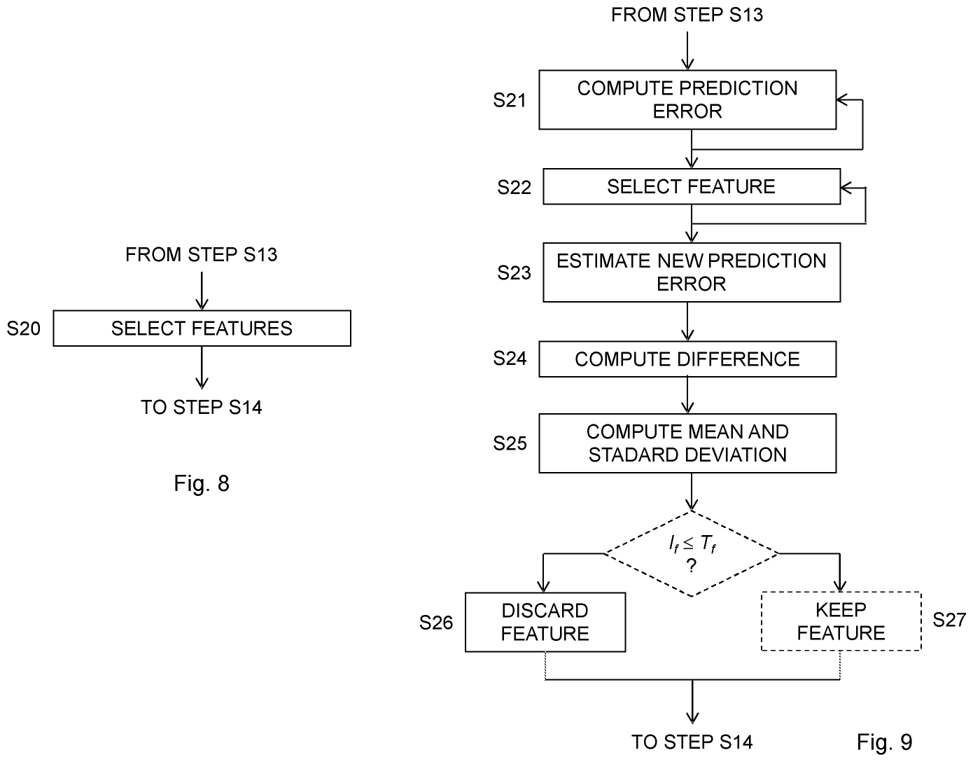

FIG. 8 is a flow chart illustrating an additional, optional step of the method shown in FIG. 7 according to an embodiment. In this embodiment, the method continues from step S13 in FIG. 7. A next step S20 comprises selecting frequency and/or time domain features among the plurality of frequency domain and time domain features to train the RF-based oxygen saturation estimation model. The method then continues to step S14 in FIG. 7, where the selected frequency domain features and/or statistical parameters of the selected time domain features are used to train the RF-based oxygen saturation estimation model.

FIG. 9 is a flow chart illustrating an embodiment of the selecting step S20 in FIG. 8. This embodiment comprises conducting steps S21 to S24 in FIG. 9 for t=1 to T. The parameter T represents a number of decision trees in the RF-based oxygen saturation estimation model. Step S21 of FIG. 9 comprises computing a prediction error Eₜ=Yₜ−Ŷₜ for a decision tree t. The parameter Yₜ represents an actual oxygen saturation value and the parameter Ŷₜ represents a prediction of the oxygen saturation value. Step S22 comprises selecting a feature f among the plurality of frequency domain and time domain features and permuting feature values until dₜᵣ=0. Step S23 comprises estimating a new prediction error E_{tf} and step S23 comprises computing a difference d_{tf}=E_{tf}−Eₜ. Hence, permutations for a particular feature f are performed until the difference d_{tf} is equal to zero. At that point, the method continues to step S25, which comprises computing a mean d_{f} and standard deviation σ_{f} over the T decision trees and computing a feature permutation importance as I_{f}=−d_{f}/σ_{f}. This feature permutation importance I_{f} is optionally compared to a threshold value T_{f}. Furthermore, the embodiment as shown in FIG. 9 comprises discarding the feature f if the feature permutation importance I_{f} is equal to lower than the threshold value T_{f} in step S26. However, if the feature permutation importance I_{f} is above the threshold value T_{f} the feature is kept in the optional step S27 and is thereby selected for usage when training the RF-based oxygen saturation estimation model.

Generally, a value of the feature permutation importance I_{f} close to zero indicates a low prediction ability of the particular feature f. Hence, frequency domain features and time domain features resulting in a feature permutation importance I_{f} well above zero generally have high prediction ability for usage by the RF-based oxygen saturation estimation model when predicting or estimating oxygen saturation based on PPG signals.

An illustrative, but non-limiting, example of a threshold value T_{f} that can be used according to the embodiments is 0.08.

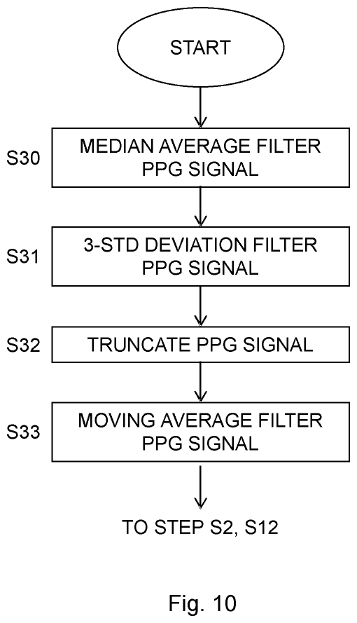

FIG. 10 is a flow chart illustrating various embodiments of pre-processing the PPG signals in step S1 in FIG. 6 and step S11 in FIG. 7.

In an embodiment, steps S1 and S11 comprise filtering the PPG signal in step S30 using a median average filter.

In a particular embodiment, this step S30 comprises filtering the PPG signal using the median average filter by sorting PPG values within a filter window in ascending order and replacing the middle PPG signal value within the filter window by the median PPG signal value within the filter window. FIG. 1B illustrates the raw PPG signal shown in FIG. 1A smoothed using such a median average filter. Hence, in an embodiment, step S30 produces a median average filtered PPG signal.

In an embodiment, steps S1 and S11 also comprise filtering the median average filtered PPG signal using a 3-standard deviation filter in step S31.

In a particular embodiment, step S31 comprises filtering the median average filtered PPG signal using the 3-standard deviation filter by calculating z-scores of data points in the median average filtered PPG signal by subtracting an average value μ_{P} of the median average filtered PPG signal P of length n from a data point Pₖ of the median average filtered PPG signal and then by dividing the output using a standard deviation σ_{P} of the median average filtered PPG signal. Step S31 also comprises, in this particular embodiment, substituting data points in the median average filtered PPG signal having a z-score higher than a threshold value T_{z} or lower than a threshold value −T_{z} by a value of a preceding data point. An illustrative, but non-limiting, example of the threshold value T_{z} is 3. FIG. 1C illustrates the 3-standard deviation filtered signal obtained by filtering the median average filtered PPG signal in FIG. 1B using a 3-standard deviation filter. Hence, in an embodiment, step S31 produces a 3-standard deviation filtered signal.

In an embodiment, steps S1 and S11 further comprise truncating the 3-standard deviation filtered signal in step S32.

In a particular embodiment, step S32 comprises truncating the part of the 3-standard deviation filtered signal between a first valley and a last valley of the 3-standard deviation filtered signal. FIG. 1D illustrates the truncated signal obtained by truncating the 3-standard deviation filtered signal shown in FIG. 1C.

In an embodiment, steps S1 and S11 additionally comprises filtering the truncated signal with a moving average filter in step S33.

In a particular embodiment, step S33 comprises filtering the truncated signal with the moving average filter by calculating smoothed signal values

$${P}_{k} = \frac{{p}_{n - k + 1} + {p}_{n - k + 2} … + {p}_{n}}{w} \\ for k = 1 … n$$

wherein k represents a data point of the truncated signal p and w is the size of a filter window. FIG. 1E illustrates the truncated signal shown in FIG. 1D filtered using a moving average filter.

In an embodiment, step S4 in FIG. 6 comprises estimating SpO₂ for the subject based on the frequency domain features and the statistical parameters of the time domain features and the oxygen saturation estimation model. In such an embodiment, the oxygen saturation estimation model is trained for estimating SpO₂ based on input frequency domain features and input statistical parameters of time domain features.

Hence, a currently preferred oxygen saturation value as estimated by the oxygen saturation estimation model is a peripheral oxygen saturation value (SpO₂), which in turn can be regarded as a representation of arterial oxygen saturation (SaO₂).

In an embodiment, steps S3 and S13 of FIGS. 6 and 7 comprise computing at least two of, preferably at least three of, more preferably at least four of, and most preferably all of mean, median, standard deviation, mean absolute deviation, and interquartile range of the time domain features. These statistical features have been shown to be relevant in order to obtain an oxygen saturation estimation model that can accurately predict oxygen saturation of a subject from a PPG signal of light reflected from the skin of the subject when illuminated by ambient light.

In an embodiment, steps S2 and S12 of FIGS. 6 and 7 comprises extracting at least two frequency domain features selected from the group consisting of amplitude of a first frequency peak of the smoothed pulse signal, frequency of the first frequency peak of the smoothed pulse signal, area under curve in the frequency range 0-2 Hz, area under the curve in the frequency range 2-5 Hz, ratio between area under curve in the frequency range 0-2 Hz and area under the curve in the frequency range 2-5 Hz, ratio between first and second frequency peaks of the smoothed pulse signal, ratio between first and third frequency peaks of the smoothed pulse signal, ratio between the frequency of the first frequency peak and the frequency of the second frequency peak of the smoothed pulse signal, ratio between the frequency of the first frequency peak and the frequency of the third frequency peak of the smoothed pulse signal, highest frequency in the smoothed pulse signal, magnitude at the highest frequency of the smoothed pulse signal, heart rate and average mean arterial pressure of the smoothed pule signal. The above mentioned group of frequency domain features is presented in Table 2 herein.

In an embodiment, steps S2 and S12 comprises extracting at least three frequency domain features selected from the group, preferably extracting at least four frequency domain features selected from the group, and more preferably extracting at least five frequency domain features selected from the group. More than five, such as six, seven, eight, nine, ten, eleven, twelve or even all thirteen frequency domain features selected from the group could be extracted in steps S2 and S12 from the smoothed pule signal.

In a particular embodiment, the group of frequency domain features consists of amplitude of a first frequency peak of the smoothed pulse signal, frequency of the first frequency peak of the smoothed pulse signal, area under curve in the frequency range 0-2 Hz, area under the curve in the frequency range 2-5 Hz, ratio between area under curve in the frequency range 0-2 Hz and area under the curve in the frequency range 2-5 Hz, ratio between first and second frequency peaks of the smoothed pulse signal, ratio between first and third frequency peaks of the smoothed pulse signal, ratio between the frequency of the first frequency peak and the frequency of the second frequency peak of the smoothed pulse signal, ratio between the frequency of the first frequency peak and the frequency of the third frequency peak of the smoothed pulse signal, highest frequency in the smoothed pulse signal, magnitude at the highest frequency of the smoothed pulse signal.

In an embodiment, steps S2 and S12 of FIGS. 6 and 7 comprises extracting at least two time domain features selected from the group consisting of the time domain features presented in Table 1.

In a particular embodiment, steps S2 and S12 comprises extracting at least two time domain features selected from the group consisting of difference between height of a peak of the smoothed pulse signal and average height of two valleys adjacent the peak, time duration between a peak of the smoothed pulse signal and a valley preceding the peak, time duration between two valleys of a pulse wave in the smoothed pulse signal, width at a selected percentage, preferably 25% or 50%, peak height between a rising branch and peak point in the smoothed pulse signal, periodic energy of the smoothed pulse signal, area under a pulse cycle in the smoothed pulse signal, time between systolic peaks and a dicrotic notch in the smoothed pulse signal, distance between diastolic valleys in the smoothed pulse signal, dicrotic notch downward curve in the smoothed pulse signal, ratio of systolic peak time to peak-to-peak interval of the smoothed pulse signal, ratio of a height of a notch to a systolic peak amplitude of the smoothed pulse signal, ratio of pulse width from right at a selected percentage, such as 75%, of systolic amplitude to notch time, time interval from a foot of the smoothed pulse signal to a time at which a first derivative of the smoothed pulse signal occurred, first maximum peak from a second derivative of the smoothed pulse signal after first maximum peak from a first derivative of the smoothed pulse signal and ratio of time interval from the foot of the smoothed signal to a time at which the first minimum peak occurred to a peak-to-peak interval of the smoothed pulse signal.

In an embodiment, steps S2 and S12 comprises extracting at least three time domain features selected from Table 1 or from the above mentioned the group, preferably extracting at least four time domain features selected from Table 1 or from the above mentioned the group, and more preferably extracting at least five time domain features selected from Table 1 or from the above mentioned the group. More than five, such as six, seven, eight, nine, ten, eleven, twelve, thirteen, fourteen, fifteen, sixteen, seventeen, eighteen, nineteen, twenty or more time domain features selected from Table 1 or from the above mentioned the group could be extracted in steps S2 and S12 from the smoothed pule signal.

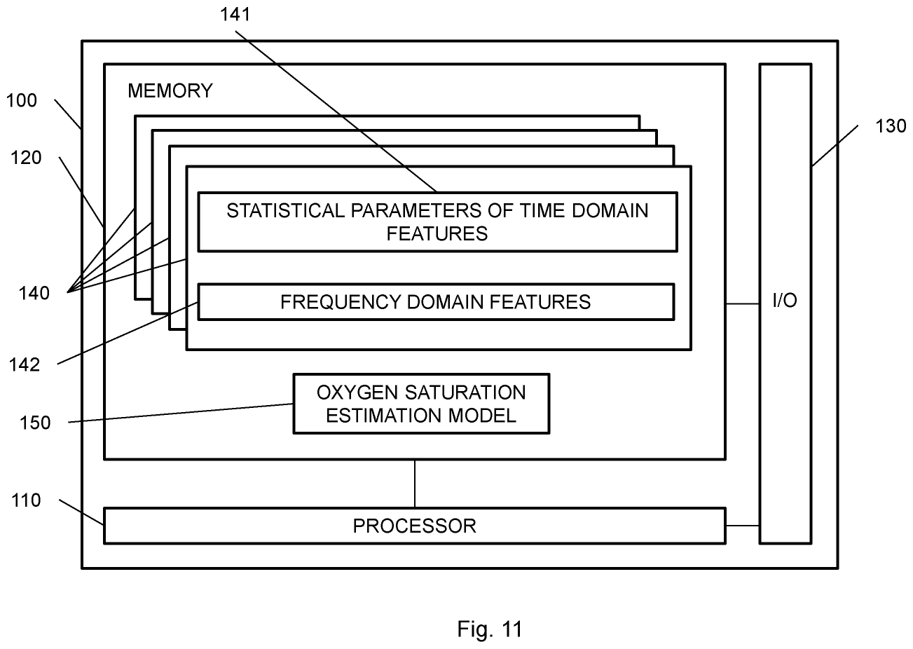

FIG. 11 is a schematic illustration of a device 100 configured to generate an oxygen saturation estimation model 150 according to an embodiment. The device 100 comprises a memory 120 configured to, at least temporarily, store sets 140 of statistical parameters of time domain features 141 and frequency domain features 142. The memory 120 also comprises the trained oxygen saturation estimation model 150. The device 100 in FIG. 11 has been shown with a single memory 120. The embodiments are, however, not limited thereto. In clear contrast, the device 100 could comprise or be, wirelessly or with wire, connected to multiple memories 120, such as memory system of multiple memories. The device 100 also comprises a processor 110 configured to process received PPG signals, extract frequency and time domain features, compute statistical parameters of time domain features and train the oxygen saturation estimation model 150 based on the input data. The device 100 further comprises a general input and output (I/O) unit 130 configured to communicate with external devices. The I/O unit 130 could represent a transmitter and receiver, or transceiver, configured to conduct wireless communication. Alternatively, or in addition, the I/O unit 130 could be configured to conduct wired communication and may then, for instance, comprise one or more input and/or output ports.

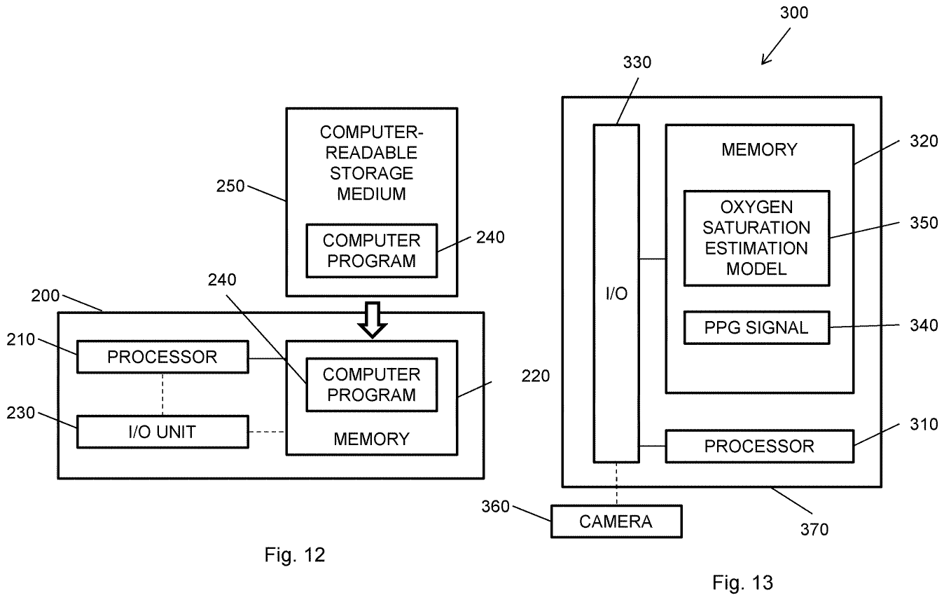

FIG. 12 is a schematic block diagram of a device 200, such as computer, comprising a processor 210 and a memory 220 that can be used to generate an oxygen saturation estimation model and/or estimate oxygen saturation using such an oxygen saturation estimation model. In such an embodiment, the training or generation and/or estimation could be implemented in a computer program 240, which is loaded into the memory 220 for execution by processing circuitry including one or more processors 210 of the device 200. The processor 120 and the memory 220 are interconnected to each other to enable normal software execution. An I/O unit 230 is preferably connected to the processor 210 and/or the memory 220 to enable reception of PPG signals.

The term processor should be interpreted in a general sense as any circuitry, system or device capable of executing program code or computer program instructions to perform a particular processing, determining or computing task. The processing circuitry including one or more processors 210 is, thus, configured to perform, when executing the computer program 240, well-defined processing tasks such as those described herein.

The processor 210 does not have to be dedicated to only execute the above-described steps, functions, procedure and/or blocks, but may also execute other tasks.

In an embodiment, the computer program 240 comprises instructions, which when executed by a processor 210, cause the processor 210 to pre-process a PPG signal of light reflected from a skin of a subject illuminated by ambient light by filtering the PPG signal to obtain a smoothed pulse signal. The processor 210 is also caused to extract a plurality of frequency domain and time domain features from the smoothed pulse signal by extracting time domain features from the smoothed pulse signal with respect to time and extracting frequency domain features from the smoothed pulse signal with respect to frequency. The processor 210 is further caused to compute statistical parameters of the time domain features. The statistical parameters represent measured quantities of a statistical population describing the respective time domain features. The processor 210 is additionally caused to estimate oxygen saturation for the subject based on the frequency domain features and the statistical parameters of the time domain features and an oxygen saturation estimation model trained for estimating oxygen saturation based on input frequency domain features and input statistical parameters of time domain features.

In another embodiment, the computer program 240 comprises instructions, which when executed by a processor 210, cause the processor 210 to pre-process a plurality of PPG signals of light reflected from skins of a plurality of subjects illuminated by ambient light by filtering the PPG signals to obtain a plurality of smoothed pulse signals. The processor 210 is also caused to extract, from each smoothed pulse signal of the plurality of smoothed pulse signals, a plurality of frequency domain and time domain features from the smoothed pulse signal by extracting time domain features from the smoothed pulse signal with respect to time and extracting frequency domain features from the smoothed pulse signal with respect to frequency. The processor 210 is further caused to compute statistical parameters of the time domain features. The statistical parameters represent measured quantities of a statistical population describing the respective time domain features. The processor 210 is additionally caused to train an oxygen saturation estimation model based on the frequency domain features and the statistical parameters of the time domain features and actual oxygen saturation values obtained for the plurality of subjects.

The proposed technology also provides a non-transitory computer-readable storage medium 250 comprising the computer program 240. By way of example, the software or computer program 240 may be realized as a computer program product, which is normally carried or stored on the non-transitory computer-readable medium 250, in particular a non-volatile medium. The non-transitory computer-readable medium 250 may include one or more removable or non-removable memory devices including, but not limited to a Read-Only Memory (ROM), a Random Access Memory (RAM), a Compact Disc (CD), a Digital Versatile Disc (DVD), a Blu-ray disc, a Universal Serial Bus (USB) memory, a Hard Disk Drive (HDD) storage device, a flash memory, a magnetic tape, or any other conventional memory device. The computer program 240 may, thus, be loaded into the operating memory 220 of the computer for execution by the processor 210 thereof.

Hence, an embodiment relates to a non-transitory computer-readable medium 250 storing instructions that, when executed by a processor 210, cause the processor 210 to pre-process a plurality of PPG signals of light reflected from skins of a plurality of subjects illuminated by ambient light by filtering the PPG signals to obtain a plurality of smoothed pulse signals. The processor 210 is also caused to extract, from each smoothed pulse signal of the plurality of smoothed pulse signals, a plurality of frequency domain and time domain features from the smoothed pulse signal by extracting time domain features from the smoothed pulse signal with respect to time and extracting frequency domain features from the smoothed pulse signal with respect to frequency. The processor 210 is further caused to compute statistical parameters of the time domain features. The statistical parameters represent measured quantities of a statistical population describing the respective time domain features. The processor 210 is additionally caused to train an oxygen saturation estimation model based on the frequency domain features and the statistical parameters of the time domain features and actual oxygen saturation values obtained for the plurality of subjects.

Another embodiment relates to a non-transitory computer-readable medium 250 storing instructions that, when executed by a processor 210, cause the processor 210 to pre-process a plurality of PPG signals of light reflected from skins of a plurality of subjects illuminated by ambient light by filtering the PPG signals to obtain a plurality of smoothed pulse signals. The processor 210 is also caused to extract, from each smoothed pulse signal of the plurality of smoothed pulse signals, a plurality of frequency domain and time domain features from the smoothed pulse signal by extracting time domain features from the smoothed pulse signal with respect to time and extracting frequency domain features from the smoothed pulse signal with respect to frequency. The processor 210 is further caused to compute statistical parameters of the time domain features. The statistical parameters represent measured quantities of a statistical population describing the respective time domain features. The processor 210 is additionally caused to train an oxygen saturation estimation model based on the frequency domain features and the statistical parameters of the time domain features and actual oxygen saturation values obtained for the plurality of subjects.

In an embodiment, the instructions cause the processor 210 to select frequency domain and/or time domain features among the plurality of frequency domain and time domain features to train a random forest based oxygen saturation estimation model. In such an embodiment, the processor 210 is caused to, for t=1 to T, wherein T represents a number of decision trees in the random forest based oxygen saturation estimation model, compute a prediction error Eₜ=Yₜ−Ŷₜ for a decision tree t, wherein Yₜ is an actual oxygen saturation value and Ŷₜ is a prediction of the oxygen saturation value; select a feature f among the plurality of frequency domain and time domain features and permuting feature values until d_{tf}=0; estimate a new prediction error E_{tf}; and compute a difference d_{tf}=E_{tf}−Eₜ. The processor 210 is also caused to compute a mean dr and standard deviation σ_{f} over the T decision trees and computing a feature permutation importance as I_{f}=−d_{f}/σ_{f} and discard the feature f if I_{f} is equal to lower than a threshold value T_{f}, wherein T_{f} is preferably 0.08.

In an embodiment, the instructions cause the processor 210 to filter the PPG signal using a median average filter. In a particular embodiment, the instructions cause the processor 210 to filter the PPG signal using the median average filter by sorting PPG signal values within a filter window in ascending order and replacing the middle PPG signal value within the filter window by the median PPG signal value within the filter window.

In an embodiment, the instructions cause the processor 210 to filter the median average filtered PPG signal using a 3-standard deviation filter. In a particular embodiment, the instructions cause the processor 210 to filter the median average filtered PPG signal using the 3-standard deviation filter by calculating z-scores of data points in the median average filtered PPG signal by subtracting an average value μ_{P} of the median average filtered PPG signal P of length n from a data point Pₖ of the median average filtered PPG signal and then by dividing the output using a standard deviation σ_{P} of the median average filtered PPG signal; and substituting data points in the median average filtered PPG signal having a z-score higher than a threshold value T_{z} or lower than a threshold value −T_{z}, wherein T_{z} is preferably 3, by a value of a preceding data point.

In an embodiment, the instructions cause the processor 210 to truncate the 3-standard deviation filtered signal. In a particular embodiment, the instructions cause the processor 210 to truncate the part of the 3-standard deviation filtered signal between a first valley and a last valley of the 3-standard deviation filtered signal.

In an embodiment, the instructions cause the processor 210 to filter the truncated signal with a moving average filter. In a particular embodiment, the instructions cause the processor 210 to filter the truncated signal with the moving average filter by calculating smoothed signal values

$${P}_{k} = \frac{{p}_{n - k + 1} + {p}_{n - k + 2} … + {p}_{n}}{w} \\ for k = 1 … n$$

wherein k represents a data point of the truncated signal p and w is the size of a filter window.

In an embodiment, the instructions cause the processor 210 to filter compute at least two of, preferably at least three of, more preferably at least four of, and most preferably all of mean, median, standard deviation, mean absolute deviation, and interquartile range of the time domain features.

In an embodiment, the instructions cause the processor 210 to extract at least two frequency domain features selected from the group consisting of amplitude of a first frequency peak of the smoothed pulse signal, frequency of the first frequency peak of the smoothed pulse signal, area under curve in the frequency range 0-2 Hz, area under the curve in the frequency range 2-5 Hz, ratio between area under curve in the frequency range 0-2 Hz and area under the curve in the frequency range 2-5 Hz, ratio between first and second frequency peaks of the smoothed pulse signal, ratio between first and third frequency peaks of the smoothed pulse signal, ratio between the frequency of the first frequency peak and the frequency of the second frequency peak of the smoothed pulse signal, ratio between the frequency of the first frequency peak and the frequency of the third frequency peak of the smoothed pulse signal, highest frequency in the smoothed pulse signal, and magnitude at the highest frequency of the smoothed pulse signal.

In an embodiment, the instructions cause the processor 210 to extract at least two time domain features selected from the group consisting of difference between height of a peak of the smoothed pulse signal and average height of two valleys adjacent the peak, time duration between a peak of the smoothed pulse signal and a valley preceding the peak, time duration between two valleys of a pulse wave in the smoothed pulse signal, width at a selected percentage, preferably 25% or 50%, peak height between a rising branch and peak point in the smoothed pulse signal, periodic energy of the smoothed pulse signal, area under a pulse cycle in the smoothed pulse signal, time between systolic peaks and a dicrotic notch in the smoothed pulse signal, distance between diastolic valleys in the smoothed pulse signal, dicrotic notch downward curve in the smoothed pulse signal, ratio of systolic peak time to peak-to-peak interval of the smoothed pulse signal, ratio of a height of a notch to a systolic peak amplitude of the smoothed pulse signal, ratio of pulse width from right at a selected percentage, such as 75%, of systolic amplitude to notch time, time interval from a foot of the smoothed pulse signal to a time at which a first derivative of the smoothed pulse signal occurred, first maximum peak from a second derivative of the smoothed pulse signal after first maximum peak from a first derivative of the smoothed pulse signal and ratio of time interval from the foot of the smoothed signal to a time at which the first minimum peak occurred to a peak-to-peak interval of the smoothed pulse signal.

The present invention also relates to a system 300 for non-contact estimation of oxygen saturation, see FIG. 13. The system 300 comprises a camera 360 configured to record a PPG signal of light reflected from a skin of a subject illuminated by ambient light. The system 300 also comprises at least one memory 320 configured to store an oxygen saturation estimation model 350 trained for estimating oxygen saturation based on input frequency domain features and input statistical parameters of time domain features. The at least one memory 320 is also configured to store the PPG signal 340 recorded by the camera 360. The system 300 further comprises at least one processor 310. The at least one processor 310 is configured to pre-process the PPG signal 340 by filtering the PPG signal 340 to obtain a smoothed pulse signal. The at least one processor 310 is also configured to extract a plurality of frequency domain and time domain features from the smoothed pulse signal by extracting time domain features from the smoothed pulse signal with respect to time and extracting frequency domain features from the smoothed pulse signal with respect to frequency and compute statistical parameters of the time domain features. The statistical parameters represent measured quantities of a statistical population describing the respective time domain features. The at least one processor 310 is further configured to estimate oxygen saturation for the subject based on the frequency domain features and the statistical parameters of the time domain features and the oxygen saturation estimation model 350 stored in the at least one memory 320.

The memory 320 and the at least one processor 310 may be implemented in a device 370, such as a computer, of the system 300. This device 370 may then be connected, wirelessly or using wires, to the camera 360 using an I/O unit 330.

The camera 360 could be any camera 360 that is able to record a PPG signal of light reflected from the skin of a subject illuminated by ambient light. The camera 360 is preferably a camera 360 capable of recording at least 100 frames per seconds, preferably at least 125 frames per seconds, such as at least 150 frames per seconds, and more preferably at least 200 frames per seconds, such as at least 250 frames per seconds or at least 300 frames per seconds. An illustrative, but non-limiting, example of a camera 360 that could be used according to the invention is a Basler MED ace camera.

### EXAMPLES

The present Examples involve development of method for estimating oxygen saturation under ambient light. The method involves training a machine learning model using features extracted from a photoplethysmography (PPG) signal recorded using a high-speed camera under ambient lighting conditions. The method comprises three main method steps: 1) pre-processing of a PPG pulse signal, 2) extraction of features from the PGG pulse signal, and 3) using features to train a random forests (RF) algorithm to estimate oxygen saturation.

### Video Recording

Subjects were video recorded using a high-speed Basler MED ace camera equipped with a Sony IMX174 complementary metal-oxide-semiconductor (CMOS) sensor, connected to a computer. Subjects were seated at a distance of one meter from the camera facing towards the camera lens. A ten-second video was recorded for each subject with a frame rate of 396.5 frames per second (fps) and an image resolution of 640×480 RGB pixels.

### Pre-Processing

Once a raw PPG signal was extracted from the recorded high-speed video using the Eulerian Magnification algorithm (FIG. 1A), the raw PPG signal was filtered using a median average filter to get a smoothed PPG signal. The median filter sorted the values of the raw PPG signal in the window in ascending order and replaced the middle value with the median value in the window. In this way, spikes in the raw PPG signal were reduced without affecting the peaks and the valleys in the raw PPG signal. The smoothed PPG signal obtained from the raw PPG pulse signal smoothed using a median average filter is shown in FIG. 1B.

The smoothed PPG signal was further filtered using a 3-standard deviation filter to reduce the height of the peaks that were abnormally high as can be observed in FIG. 1B. In a first step, a 3-standard deviation filter was applied to the smoothed PPG signal. This was done by calculating the z-scores of the data points in the smoothed PPG signal. A z-score is the number of standard deviations by which a data point is above or below the average value of the smoothed PPG pulse signal P. The z-score Zₖ was computed by subtracting the average value of the smoothed PPG signal P of length n, given as μ_{P}, from an individual data point of the smoothed PPG signal, given as Pₖ, and then by dividing the output using the standard deviation op of the smoothed PPG signal (equation 1).

$${Z}_{k} = \frac{{P}_{k} - {μ}_{P}}{{σ}_{P}} \\ for k = 1 … n \qquad ( 1 )$$

Once the z-score was calculated, the data points in the smoothed PPG signal that had a z-score higher than 3 and lower than −3 were substituted by the value of the previous data point consequently reducing spikes in the smoothed PPG pulse signal as shown in FIG. 1C.

Once filtered, the smoothed PPG signal was truncated by keeping the part of the filtered and smoothed PPG signal that lied between the first and the last valleys of the filtered and smoothed PPG signal. The first and last peaks of the filtered and smoothed PPG signal were removed because the initial and the end of the filtered and smoothed PPG signal may consist of movements of the subject to get into position for recording. This was done by applying a valley finder algorithm and the part of the filtered and smoothed PPG signal between the second and the second-last valley was selected for further processing. The truncated PPG signal is shown in FIG. 1D.

The truncated PPG signal was further smoothed using a moving average filter to remove signal aberrations. A moving average filter, also referred to as a rolling average filter, creates a series of averages of values of samples within a window, that then rolls over to the full dataset to smooth out short-term fluctuations. The moving average filter for the truncated PPG pulse signal p of length n is given in equation 2.

$${P}_{k} = \frac{{p}_{n - k + 1} + {p}_{n - k + 2} … + {p}_{n}}{w} \\ for k = 1 … n \qquad ( 2 )$$

where k is the data point of the truncated PPG signal p and w is the size of the window. The final PPG pulse signal P is shown in FIG. 1E. This final PPG signal was used for feature extraction.

### Feature Extraction

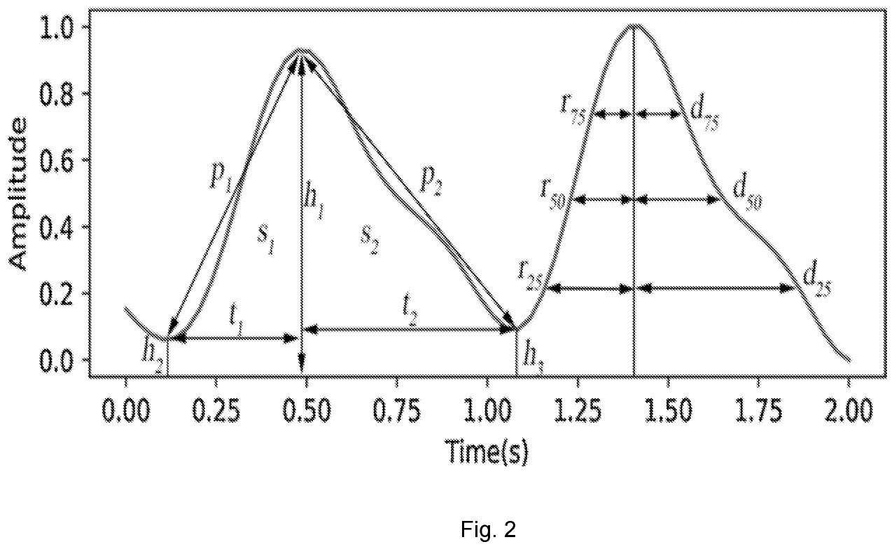

A total of 493 frequency and time domain features were extracted from the final PPG signal P. Statistical parameters of time domain features, shown in FIG. 2 and including mean, median, standard deviation, mean absolute deviation, and interquartile range, were computed. Time domain features are given in Table 1 and frequency domain features are given in Table 2.

TABLE 1
Time domain features for estimating oxygen saturation. Statistical parameters, such as mean, standard
deviation, median, mean absolute deviation, and interquartile range were computed for each feature.
Total features = 96 features × 5 statistical parameters = 480 time domain features.

| No. | Feature | Description |
|---|---|---|
| 1 | h1 | Height of the peak of a pulse wave |
| 2 | (h2 + h3)/2 | The average height of two subsequent valleys in a pulse wave. |
| 3 | h1(c) − ((h2(c) + h3(c))/2) | Difference between the height of the peak of the pulse wave |
| | | and the average height of two adjacent valleys |
| 4 | t1 | The time duration between a peak and the valley on the left of |
| | | the peak |
| 5 | t2 | The time duration between a peak and the valley on the right of |
| | | the peak |
| 6 | t1 + t2 | The time duration between two valleys of a pulse wave |
| 7 | t1 − t2 | Time difference between two valleys of a pulse wave |
| 8 | t1/t2 | Time ratio between two valleys of a pulse wave |
| 9 | s1 | Area of the rising branch of waveform |
| 10 | s2 | Area of the falling branch of waveform |
| 11 | s1 + s2 | The total area under the pulse wave |
| 12 | s1/(s1 + s2) | The ratio of the area of the rising branch and the total area |
| | | under the pulse waveform |
| 13 | s2/(s1 + s2) | The ratio of the area of the falling branch and the total area |
| | | under the pulse waveform |
| 14 | s1/s2 | The ratio of the area of the rising branch and the falling branch |
| | | of the pulse waveform |
| 15 | p1 | The slope of the rising branch of a pulse waveform |
| 16 | p2 | The slope of the falling branch of a pulse waveform |
| 17 | Lt_wt25 | Width at 25% peak height between a rising branch and peak |
| | | point |
| 18 | Lt_wt50 | Width at 50% peak height between the rising branch and peak |
| | | point |
| 19 | Lt_wt75 | Width at 75% peak height between the rising branch and peak |
| | | point |
| 20 | Rt_wt25 | Width at 25% peak height between the falling branch and peak |
| | | point |
| 21 | Rt_wt50 | Width at 50% peak height between the falling branch and peak |
| | | point |
| 22 | Rt_wt75 | Width at 25% peak height between the falling branch and peak |
| | | point |
| 23 | KTE | Instantaneous waveform energy using Teager Energy Operator |
| 24 | K | Characteristic quantify of a pulse waveform |
| 25 | En | Periodic energy of pulse waveform |
| 26 | Cycle area | The area under a pulse cycle |
| 27 | PTT | Pulse transit time in a pulse cycle |
| 28 | Cycle MAP | Mean arterial pressure in a cycle |
| 29 | Pk | Systolic peak height |
| 30 | Dk | Diastolic valley height |
| 31 | Pdias | The area under dicrotic notch and diastolic valley |
| 32 | Psys | The area under systolic peak and diastolic notch |
| 33 | KTE_signal | Instantaneous signal energy using Teager Energy Operator |
| 34 | Lt_ht25 | Height at 25% of the total time of the rising branch of a |
| | | waveform |
| 35 | Lt_ht50 | Height at 50% of the total time of the rising branch of a |
| | | waveform |
| 36 | Lt_ht75 | Height at 75% of the total time of the rising branch of a |
| | | waveform |
| 37 | Rt_ht25 | Height at 25% of the total time of the falling branch of a |
| | | waveform |
| 38 | Rt_ht50 | Height at 50% of the total time of the falling branch of a |
| | | waveform |
| 39 | Rt_ht75 | Height at 75% of the total time of the falling branch of a |
| | | waveform |
| 40 | NT | Dicrotic notch time - Time between the locations of the first |
| | | diastolic valley of the waveform and dicrotic notch |
| 41 | NH | Dicrotic notch height |
| 42 | PPI | The time between systolic peaks and dicrotic notch in a pulse |
| | | waveform |
| 43 | Pk_time(2) − Pk_time(1) | Distance between systolic peaks |
| 44 | DV_time (2) − DV_time (1) | Distance between diastolic valleys |
| 45 | A1/A2 | Inflection point area - A1 is the area under the first diastolic |
| | | valley and the dicrotic notch. A2 is the area under the dicrotic |
| | | notch and the second diastolic valley in a pulse waveform. |
| 46 | NH/Pk | Augmentation index - a ratio of dicrotic notch height and |
| | | systolic peak height |
| 47 | (Pk − NH)/Pk | Alternative Augmentation Index - Difference between systolic |
| | | and dicrotic notch height divided by systolic peak height |
| 48 | t1/Pk | Systolic peak output curve - The ratio of systolic peak time to |
| | | systolic peak amplitude |
| 49 | NH/((t1 − t2) − (t1 − NT)) | Dicrotic notch downward curve - The ratio of diastolic peak |
| | | amplitude to the differences between pulse interval and height |
| | | of notch time |
| 50 | (Pk_time(1) − DV_time(1))/ | The ratio of systolic peak time to the peak-to-peak interval of |
| | (Pk_time(2) − Pk_time(1)) | the PPG waveform, Pk: Systolic peak, DV: Diastolic valley |
| 51 | (NT − DV_time(1))/ | The ratio of dicrotic notch time to the peak-to-peak interval of |
| | (Pk_time (2) − Pk_time(1)) | the PPG waveform |
| 52 | (NT − Pk_time(1))/ | The ratio of dicrotic notch time to the peak-to-peak interval of |
| | (Pk_time (2) − Pk_time(1)) | the PPG waveform |
| 53 | NH/Pk | The ratio of the height of notch to the systolic peak amplitude |
| 54 | (NT − DV_time(1))/NH | The ratio of the notch time to the height of the notch |
| 55 | Pk/((DV_time (2) − DV_time | The ratio of systolic peak amplitude to the difference between |
| | (1)) − (Pk_time (1) − | pulse interval and systolic peak time |
| | DV_time (1))) | |
| 56 | NH/((DV_time (2) − DV_time | The ratio of the height of notch to the difference between pulse |
| | (1)) − (NT − DV_time (1))) | interval and notch time |
| 57 | Lt_wt25/(Pk_time (1) − | The ratio of pulse width from the left at 25% of systolic |
| | DV_time (1)) | amplitude to systolic peak time |
| 58 | Rt_wt25/(Pk_time (1) − | The ratio of pulse width from right at 25% of systolic amplitude |
| | DV_time (1)) | to systolic peak time |
| 59 | Lt_wt25/(NT − | The ratio of pulse width from the left at 25% of systolic |
| | DV_time (1)) | amplitude to notch time |
| 60 | Rt_wt25/(NT − | The ratio of pulse width from right at 25% of systolic amplitude |
| | DV_time (1)) | to notch time |
| 61 | Lt_wt25/(NT − | The ratio of pulse width from the left at 25% of systolic |
| | Pk_time (1)) | amplitude to DT |
| 62 | Rt_wt25/(NT − | The ratio of pulse width from right at 25% of systolic amplitude |
| | Pk_time (1)) | to notch time |
| 63 | Lt_wt25/(DV_time (2) − | The ratio of pulse width from the left at 25% of systolic |
| | DV_time (1)) | amplitude to pulse interval |
| 64 | Rt_wt25/(DV_time (2) − | The ratio of pulse width from right at 25% of systolic amplitude |
| | DV_time (1)) | to pulse interval |
| 65 | Lt_wt50/(Pk_time (1) − | The ratio of pulse width from the left at 50% of systolic |
| | DV_time (1)) | amplitude to systolic peak time |
| 66 | Rt_wt50/(Pk_time (1) − | The ratio of pulse width from right at 50% of systolic amplitude |
| | DV_time (1)) | to systolic peak time |
| 67 | Lt_wt50/(NT − | The ratio of pulse width from the left at 50% of systolic |
| | DV_time (1)) | amplitude to notch time |
| 68 | Rt_wt50/(NT − | The ratio of pulse width from right at 50% of systolic amplitude |
| | Pk_time (1)) | to DT |
| 69 | Lt_wt50/(NT − | The ratio of pulse width from the left at 50% of systolic |
| | Pk_time (1)) | amplitude to DT |
| 70 | Rt_wt50/(NT − | The ratio of pulse width from right at 50% of systolic amplitude |
| | Pk_time (1)) | to DT |
| 71 | Lt_wt50/(DV_time (2) − | The ratio of pulse width from the left at 50% of systolic |
| | DV_time (1)) | amplitude to pulse interval |
| 72 | Rt_wt50/(DV_time (2) − | The ratio of pulse width from right at 50% of systolic amplitude |
| | DV_time (1)) | to pulse interval |
| 73 | Lt_wt75/(Pk_time (1) − | The ratio of pulse width from the left at 75% of systolic |
| | DV_time (1)) | amplitude to systolic peak time |
| 74 | Rt_wt75/(Pk_time (1) − | The ratio of pulse width from right at 75% of systolic amplitude |
| | DV_time (1)) | to systolic peak time |
| 75 | Lt_wt75/(NT − | The ratio of pulse width from the left at 75% of systolic |
| | DV_time (1)) | amplitude to notch time |
| 76 | Rt_wt75/(NT − | The ratio of pulse width from right at 75% of systolic amplitude |
| | DV_time (1)) | to notch time |
| 77 | Lt_wt75/(NT − | The ratio of pulse width from the left at 75% of systolic |
| | Pk_time (1)) | amplitude to DT |
| 78 | Rt_wt75/(NT − | The ratio of pulse width from right at 75% of systolic amplitude |
| | Pk_time (1)) | to DT |
| 79 | Lt_wt75/(DV_time (2) − | The ratio of pulse width from the left at 75% of systolic |
| | DV_time (1)) | amplitude to pulse interval |
| 80 | Rt_wt75/(DV_time (2) − | The ratio of pulse width from right at 75% of systolic amplitude |
| | DV_time (1)) | to pulse interval |
| 81 | a1 | The first maximum peak from the first derivative of the PPG |
| | | waveform |
| 82 | ta1 | The time interval from the foot of the PPG waveform to the time |
| | | at which the first derivative of the PPG waveform occurred |
| 83 | a2 | The first maximum peak from the second derivative of the PPG |
| | | waveform after a1 |
| 84 | ta2 | The time interval from the foot of the PPG waveform |
| | | to the time at which the first maximum peak from the second |
| | | derivative of the PPG occurred |
| 85 | b1 | The first minimum peak from the first derivative of the PPG |
| | | waveform after the first maximum peak |
| 86 | tb1 | The time interval from the foot of the PPG waveform to the time |
| | | at which the first minimum peak occurred |
| 87 | b2 | The first minimum peak from the second derivative of the PPG |
| | | waveform |
| 88 | tb2 | The time interval from the foot of the PPG waveform to the time |
| | | at which b2 |
| 89 | b2/a2 | The ratio of b2 to a2 |
| 90 | b1/a1 | The ratio of the first minimum peak of the first derivative after |
| | | a1 to the first maximum peak of the first derivative |
| 91 | ta1/(Pk_time (2) − | The ratio of ta1 to the peak-to-peak interval of the PPG |
| | Pk_time(1)) | waveform |
| 92 | ta2/(Pk_time (2) − | The ratio of ta2 to the peak-to-peak interval of the PPG |
| | Pk_time(1)) | waveform |
| 93 | tb1/(Pk_time (2) − | The ratio of tb1 to the peak-to-peak interval of the PPG |
| | Pk_time(1)) | waveform |
| 94 | tb2/(Pk_time (2) − | The ratio of tb2 to the peak-to-peak interval of the PPG |
| | Pk_time(1)) | waveform |
| 95 | (ta1 − ta2)/(Pk_time (2) − | The ratio of the difference between ta1 and ta2 to the peak-to- |
| | Pk_time(1)) | peak interval of the PPG waveform |
| 96 | (tb1 − tb2)/(Pk_time (2) − | The ratio of the difference between tb1 and tb2 to the peak-to- |
| | Pk_time(1)) | peak interval of the PPG waveform |

TABLE 2
Frequency features for estimating oxygen saturation.

| No. | Feature | Description |
|---|---|---|
| 97 | Peak-1 | The amplitude of the first frequency peak of the pulse signal. |
| 98 | Freq-1 | The frequency at the first peak of the pulse signal. |
| 99 | A0-2 | The area under the curve in the frequency range 0-2 Hz |
| 100 | A2-5 | The area under the curve in the frequency range 2-5 Hz |
| 101 | A0-2/A2-5 | Ratio between A0-2 and A2-5 |
| 102 | Peak-1/Peak-2 | Ratio between first and seconds frequency peaks of the pulse signal |
| 103 | Peak-1/Peak-3 | The ratio between the first and third frequency peaks of the pulse signal |
| 104 | Freq-1/Freq-2 | The ratio between the frequency at the first peak and the |
| | | frequency at the second peak of the pulse signal |
| 105 | Freq-1/Freq-3 | The ratio between the frequency at the first peak and the |
| | | frequency at the third peak of the pulse signal |
| 106 | Fmax | The highest frequency in the pulse signal |
| 107 | mag_Fmax | Magnitude at the highest frequency of the pulse signal |
| 108 | HR | Heart rate |
| 109 | MAP | Average mean arterial pressure of a pulse signal |

### Training Random Forests for Estimating Oxygen Saturation

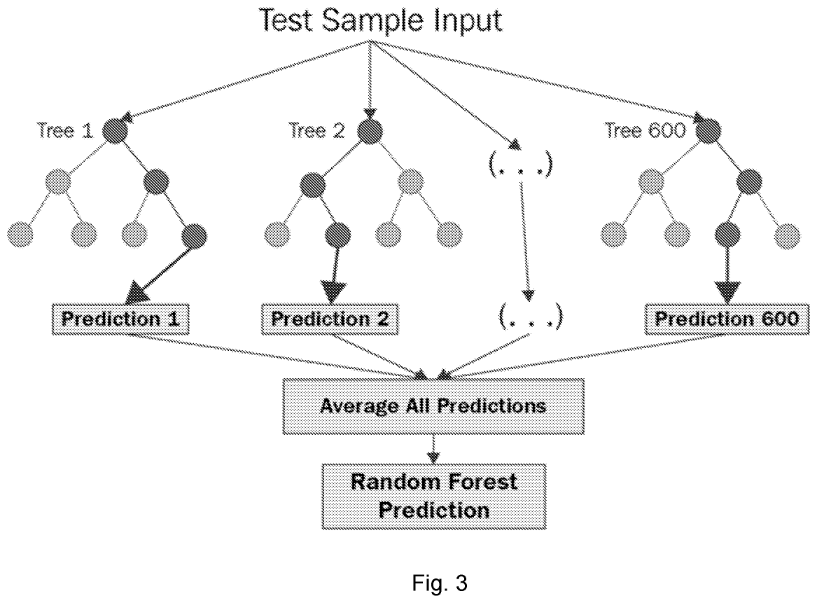

Random forests (RF) are an ensemble-based method of machine learning. An RF algorithm operates by dividing the training data into random subsets and training multiple decision trees by using these subsets through a process called Bagging. Bagging splits training data in a way that two-thirds of the data that is randomly selected from the full training set is used for training a decision tree in the forests. The rest of the one-third of the data is used for testing that decision tree. The test data are termed out-of-bag (OOB) samples. An error in predicting an i^{th} OOB sample is computed using equation 3.

$${E}_{i} [ Y ] = {Y}_{i} - {\hat{Y}}_{i} \qquad ( 3 )$$

where Yᵢ is the actual value of the OOB sample, and Ŷᵢ is the prediction of the OOB sample by an i^{th} decision tree. An average value of predictions produced by all the decision trees in the forests is the prediction from the model as shown in FIG. 3.

For oxygen saturation estimation, which is a regression problem, the overall performance of the RF algorithm was analyzed based on the R² coefficient computed using equation 4.

$${R}^{2} = 1 - \frac{{\sum }_{i} {( {Y}_{i} - {\hat{Y}}_{i} )}^{2}}{{\sum }_{i} {( {Y}_{i} - E [ Y ] )}^{2}} \qquad ( 4 )$$

where E[Y] is the average OOB prediction error. By using multiple decision trees for prediction, the algorithm eliminates prediction bias that occurs if a single decision tree is used for decision making. Also, the random selection of data for training and testing reduces variance in the data that prevents overfitting.

Another advantage of using the RF algorithm is that it performs feature selection during training. Features that are most correlated with the training targets are selected by the RF algorithm using permutation scores. RF permutes feature values to estimate if the permutation deteriorates the prediction performance compared to a baseline. The features that are not correlated show no changes when the values are permutated suggesting that there is no difference between the permuted values and the original sequence of values. This suggests that the feature is a noise that does not contribute to training and can be discarded. On the other hand, the permutation of features that are correlated with the training targets results in reducing the prediction accuracy.

A feature's permutation score was computed as follows:

```
for an RF with a total of T decision trees and a total number of F features
  for t = 1 to T
    compute the baseline OOB prediction error Eₜ for a tree t;
    select a feature f and permute feature values;
    estimate a new OOB prediction error E_{tf};
    compute the difference between the baseline and new prediction error using d_{tf} = E_{tf} − Eₜ;
    if d_{tf} =0
      stop permutations;
  end
  compute mean d_{f} and standard deviation σ_{f} over T trees;
  feature permutation importance is computed as l_{f} = −d_{f}/σ_{f};
end
```

A value of I_{f} equals or near to 0 suggests low prediction ability of feature f.

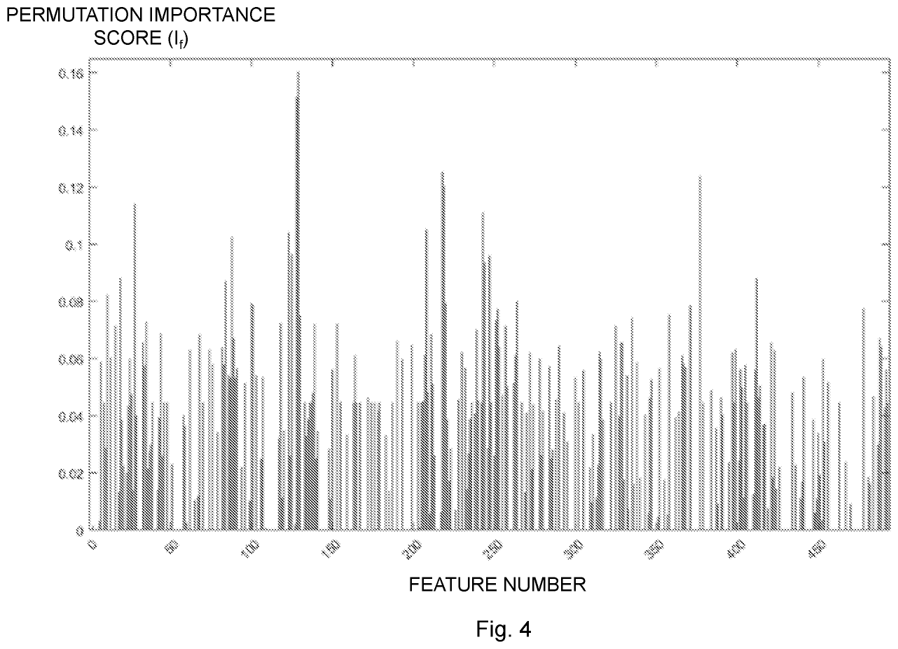

Twenty features produced permutation importance scores above 0.08 as shown in FIG. 4. These features were selected for training the RF algorithm. A list of the selected features is given in Table 3.

TABLE 3
Selected features for training the random forests

| No. | Feature |
|---|---|
| 1 | Mean of feature number 3 |
| 2 | Mean absolute deviation of feature number 4 |
| 3 | The standard deviation of feature number 6 |
| 4 | Mean absolute deviation of feature number 17 |
| 5 | The standard deviation of feature number 18 |
| 6 | The standard deviation of feature number 25 |
| 7 | The interquartile range of feature number 25 |
| 8 | The standard deviation of feature number 26 |
| 9 | Mean absolute deviation of feature number 26 |
| 10 | The standard deviation of feature number 42 |
| 11 | The standard deviation of feature number 44 |
| 12 | Mean absolute deviation of feature number F44 |
| 13 | The standard deviation of feature number 49 |
| 14 | Mean absolute deviation of feature number 49 |
| 15 | Median of feature number 50 |
| 16 | Mean absolute deviation of feature number 53 |
| 17 | Median of feature number 76 |
| 18 | The standard deviation of feature number 82 |
| 19 | Median of feature number 83 |
| 20 | The interquartile range of feature number 93 |

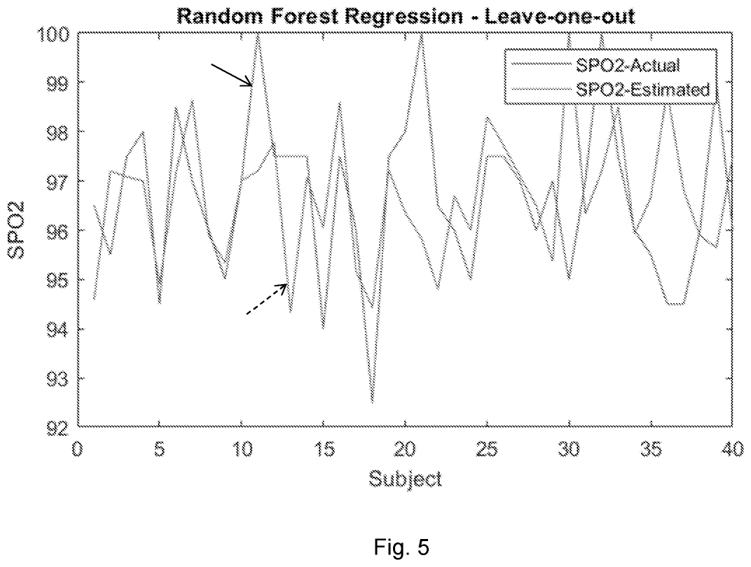

Finally, the model performance based on a Leave one out cross-validation using 50 samples is shown in FIG. 5.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-18. (canceled)

19. A method for non-contact estimation of oxygen saturation, the method comprising:
pre-processing a photoplethysmography (PPG) signal of light reflected from a skin of a subject illuminated by ambient light by filtering the PPG signal to obtain a smoothed pulse signal;
extracting a plurality of frequency domain and time domain features from the smoothed pulse signal by extracting time domain features from the smoothed pulse signal with respect to time and extracting frequency domain features from the smoothed pulse signal with respect to frequency;
computing statistical parameters of the time domain features, wherein the statistical parameters represent measured quantities of a statistical population describing the respective time domain features; and
estimating oxygen saturation for the subject based on the frequency domain features and the statistical parameters of the time domain features and an oxygen saturation estimation model trained for estimating oxygen saturation based on input frequency domain features and input statistical parameters of time domain features.

20. The method according to claim 19, wherein pre-processing comprises filtering the PPG signal using a median average filter.

21. The method according to claim 20, wherein filtering the PPG signal comprises filtering the PPG signal using the median average filter by sorting PPG signal values within a filter window in ascending order and replacing the middle PPG signal value within the filter window by the median PPG signal value within the filter window.

22. The method according to claim 20, wherein pre-processing further comprises filtering the median average filtered PPG signal using a 3-standard deviation filter.

23. The method according to claim 22, wherein filtering the median average filtered PPG signal comprises filtering the median average filtered PPG signal using the 3-standard deviation filter by
calculating z-scores of data points in the median average filtered PPG signal by subtracting an average value μ_{P} of the median average filtered PPG signal P of length n from a data point Pₖ of the median average filtered PPG signal and then by dividing the output using a standard deviation σ_{P} of the median average filtered PPG signal; and
substituting data points in the median average filtered PPG signal having a z-score higher than a threshold value T_{z} or lower than a threshold value −T_{z} by a value of a preceding data point.

24. The method according to claim 23, wherein pre-processing further comprises truncating the 3-standard deviation filtered signal.

25. The method according to claim 24, wherein truncating the 3-standard deviation filtered signal comprises truncating the part of the 3-standard deviation filtered signal between a first valley and a last valley of the 3-standard deviation filtered signal.

26. The method according to claim 24, wherein pre-processing further comprises filtering (S33) the truncated signal with a moving average filter.

27. The method according to claim 26, wherein filtering the truncated signal comprises filtering the truncated signal with the moving average filter by calculating smoothed signal values
$${P}_{k} = \frac{{p}_{n - k + 1} + {p}_{n - k + 2} … + {p}_{n}}{w} \\ for k = 1 … n$$
wherein k represents a data point of the truncated signal p and w is the size of a filter window.

28. The method according to claim 19, wherein computing statistical parameters comprises computing at least two of mean, median, standard deviation, mean absolute deviation, and interquartile range of the time domain features.

29. The method according to claim 19, wherein extracting a plurality of frequency domain features comprises extracting at least two frequency domain features selected from the group consisting of amplitude of a first frequency peak of the smoothed pulse signal, frequency of the first frequency peak of the smoothed pulse signal, area under curve in the frequency range 0-2 Hz, area under the curve in the frequency range 2-5 Hz, ratio between area under curve in the frequency range 0-2 Hz and area under the curve in the frequency range 2-5 Hz, ratio between first and second frequency peaks of the smoothed pulse signal, ratio between first and third frequency peaks of the smoothed pulse signal, ratio between the frequency of the first frequency peak and the frequency of the second frequency peak of the smoothed pulse signal, ratio between the frequency of the first frequency peak and the frequency of the third frequency peak of the smoothed pulse signal, highest frequency in the smoothed pulse signal, and magnitude at the highest frequency of the smoothed pulse signal.

30. The method according to claim 19, wherein extracting a plurality of time domain features comprises extracting at least two time domain features selected from the group consisting of difference between height of a peak of the smoothed pulse signal and average height of two valleys adjacent the peak, time duration between a peak of the smoothed pulse signal and a valley preceding the peak, time duration between two valleys of a pulse wave in the smoothed pulse signal, width at a selected percentage, preferably 25% or 50%, peak height between a rising branch and peak point in the smoothed pulse signal, periodic energy of the smoothed pulse signal, area under a pulse cycle in the smoothed pulse signal, time between systolic peaks and a dicrotic notch in the smoothed pulse signal, distance between diastolic valleys in the smoothed pulse signal, dicrotic notch downward curve in the smoothed pulse signal, ratio of systolic peak time to peak-to-peak interval of the smoothed pulse signal, ratio of a height of a notch to a systolic peak amplitude of the smoothed pulse signal, ratio of pulse width from right at a selected percentage, such as 75%, of systolic amplitude to notch time, time interval from a foot of the smoothed pulse signal to a time at which a first derivative of the smoothed pulse signal occurred, first maximum peak from a second derivative of the smoothed pulse signal after first maximum peak from a first derivative of the smoothed pulse signal and ratio of time interval from the foot of the smoothed signal to a time at which the first minimum peak occurred to a peak-to-peak interval of the smoothed pulse signal.

31. A computer-implemented method of generating an oxygen saturation estimation model, the method comprising:
pre-processing a plurality of photoplethysmography (PPG) signals of light reflected from skins of a plurality of subjects illuminated by ambient light by filtering the PPG signals to obtain a plurality of smoothed pulse signals;
extracting, from each smoothed pulse signal of the plurality of smoothed pulse signals, a plurality of frequency domain and time domain features from the smoothed pulse signal by extracting time domain features from the smoothed pulse signal with respect to time and extracting frequency domain features from the smoothed pulse signal with respect to frequency;
computing statistical parameters of the time domain features, wherein the statistical parameters represent measured quantities of a statistical population describing the respective time domain features; and
training the oxygen saturation estimation model based on the frequency domain features and the statistical parameters of the time domain features and actual oxygen saturation values obtained for the plurality of subjects.

32. The method according to claim 31, wherein the oxygen saturation estimation model is a random forest based oxygen saturation estimation model.

33. The method according to claim 32, wherein training the random forest based oxygen saturation estimation model comprises:
selecting frequency domain and/or time domain features among the plurality of frequency domain and time domain features to train the random forest based oxygen saturation estimation model by:
for t=1 to T, wherein T represents a number of decision trees in the random forest based oxygen saturation estimation model,
computing a prediction error Eₜ=Yₜ−Ŷₜ for a decision tree t, wherein Yₜ is an actual oxygen saturation value and Ŷₜ is a prediction of the oxygen saturation value;
selecting a feature f among the plurality of frequency domain and time domain features and permuting feature values until d_{tf}=0;
estimating a new prediction error E_{tf};
computing a difference d_{tf}=E_{tf}−Eₜ;
computing a mean dr and standard deviation σ_{f} over the T decision trees and computing a feature permutation importance as I_{f}=−d_{f}/σ_{f}; and
discarding the feature f if I_{f} is equal to lower than a threshold value T_{f}.

34. The method according to claim 31, wherein computing statistical parameters comprises computing at least two of mean, median, standard deviation, mean absolute deviation, and interquartile range of the time domain features.

35. The method according to claim 31, wherein extracting a plurality of frequency domain features comprises extracting at least two frequency domain features selected from the group consisting of amplitude of a first frequency peak of the smoothed pulse signal, frequency of the first frequency peak of the smoothed pulse signal, area under curve in the frequency range 0-2 Hz, area under the curve in the frequency range 2-5 Hz, ratio between area under curve in the frequency range 0-2 Hz and area under the curve in the frequency range 2-5 Hz, ratio between first and second frequency peaks of the smoothed pulse signal, ratio between first and third frequency peaks of the smoothed pulse signal, ratio between the frequency of the first frequency peak and the frequency of the second frequency peak of the smoothed pulse signal, ratio between the frequency of the first frequency peak and the frequency of the third frequency peak of the smoothed pulse signal, highest frequency in the smoothed pulse signal, and magnitude at the highest frequency of the smoothed pulse signal.

36. The method according to claim 31, wherein extracting a plurality of time domain features comprises extracting at least two time domain features selected from the group consisting of difference between height of a peak of the smoothed pulse signal and average height of two valleys adjacent the peak, time duration between a peak of the smoothed pulse signal and a valley preceding the peak, time duration between two valleys of a pulse wave in the smoothed pulse signal, width at a selected percentage, preferably 25% or 50%, peak height between a rising branch and peak point in the smoothed pulse signal, periodic energy of the smoothed pulse signal, area under a pulse cycle in the smoothed pulse signal, time between systolic peaks and a dicrotic notch in the smoothed pulse signal, distance between diastolic valleys in the smoothed pulse signal, dicrotic notch downward curve in the smoothed pulse signal, ratio of systolic peak time to peak-to-peak interval of the smoothed pulse signal, ratio of a height of a notch to a systolic peak amplitude of the smoothed pulse signal, ratio of pulse width from right at a selected percentage, such as 75%, of systolic amplitude to notch time, time interval from a foot of the smoothed pulse signal to a time at which a first derivative of the smoothed pulse signal occurred, first maximum peak from a second derivative of the smoothed pulse signal after first maximum peak from a first derivative of the smoothed pulse signal and ratio of time interval from the foot of the smoothed signal to a time at which the first minimum peak occurred to a peak-to-peak interval of the smoothed pulse signal.

37. A system for non-contact estimation of oxygen saturation, the system comprising:
a camera configured to record a photoplethysmography (PPG) signal of light reflected from a skin of a subject illuminated by ambient light;
at least one memory configured to store:
an oxygen saturation estimation model trained for estimating oxygen saturation based on input frequency domain features and input statistical parameters of the domain features; and
the PPG signal recorded by the camera; and
at least one processor configured to:
pre-process the PPG signal by filtering the PPG signal to obtain a smoothed pulse signal;
extract a plurality of frequency domain and time domain features from the smoothed pulse signal by extracting time domain features from the smoothed pulse signal with respect to time and extracting frequency domain features from the smoothed pulse signal with respect to frequency;
compute statistical parameters of the time domain features, wherein the statistical parameters represent measured quantities of a statistical population describing the respective time domain features; and
estimate oxygen saturation for the subject based on the frequency domain features and the statistical parameters of the time domain features and the oxygen saturation estimation model stored in the at least one memory.
